(19) United States
(12) Patent Application Publication
(10) Pub. No.: US 2006/0015020 A1
(43) Pub. Date: Jan. 19, 2006

(54) SYSTEMS AND METHODS FOR MANUFACTURE OF AN ANALYTE-MEASURING DEVICE INCLUDING A MEMBRANE SYSTEM

(75) Inventors: Paul Neale, San Diego, CA (US); Mark A. Tapsak, Orangeville, PA (US); Sean T. Saint, San Diego, CA (US); James R. Petisce, San Diego, CA (US); James H. Brauker, San Diego, CA (US); Mark Brister, Encinitas, CA (US)

Correspondence Address: KNOBBE MARTENS OLSON & BEAR LLP, 2040 MAIN STREET, FOURTEENTH FLOOR, IRVINE, CA, 92614, US

(73) Assignee: DexCom, Inc., San Diego, CA (US)

(21) Appl. No.: 10/885,476

(22) Filed: Jul. 6, 2004

(51) Int. Cl. A61B 5/00; B29C 65/00

(52) U.S. Cl. 600/309; 600/365; 156/60

(57) ABSTRACT

Abstract of the Disclosure

Systems and methods for manufacture of an analyte-measuring device, including adhering a membrane system that allows the passage of the analyte therethrough to a sensing mechanism. The implantable analyte-measuring device includes a body formed from a material that is substantially similar to the membrane system so as to enable sufficiently strong adhesion therebetween, which enables a sufficiently strong adhesive joint capable of withstanding in vivo cellular forces. In some embodiments, the device body includes an insert to which the membrane system is adhered, wherein the insert is formed from a material substantially similar to the membrane system to enable strong adhesion therebetween. The analyte-measuring device is designed with optimized device sizing and maximum membrane adhesion and longevity to enable controlled transport of analytes through the membrane system in vivo with improved device performance.

### Detailed Description of the Invention

### Field of the Invention

The present invention relates generally to the systems and methods associated with an analyte-measuring device that measures a concentration of analyte of interest or a substance indicative of the concentration or presence of the analyte.

### Background of the Invention

A variety of analyte-measuring devices have been developed in the past few decades for measuring a variety of analytes. Some analyte-measuring devices are substantially continuous devices, while others can analyze a plurality of intermittent blood samples. Some analyte-measuring devices are subcutaneous, transdermal, or intravascular devices, which are typically invasive or minimally invasive, while others are non-invasive in nature. The measurement techniques used by these devices include enzymatic, chemical, physical, electrochemical, spectrophotometric, polarimetric, calorimetric, radiometric, and the like, and generally provide an output signal indicative of the concentration of the analyte of interest. The output signal is typically a raw signal that is used to provide a useful value of the analyte of interest to a user, such as a patient or doctor, using the device. Typically, these analyte-measuring devices include a membrane system that functions to control the flux of a biological fluid therethrough and/or to protect sensitive regions of the device from contamination by the biological fluid, for example. Conventional analyte-measuring devices that use a variety of techniques to manufacture the device, including the incorporation of a membrane system, however, suffer from a variety of disadvantages.

### Summary of the Invention

The preferred embodiments provide systems and methods for manufacturing an analyte-measuring device, including a membrane system, that minimize the size of the device and maximize adhesion and longevity of the membrane to the device.

Accordingly, in a first embodiment an implantable analyte-measuring device is provided, including a sensor body formed from a first material, wherein the sensor body includes a sensing region for measuring an analyte; and a membrane system configured to permit passage of the analyte at least partially therethrough, wherein the membrane system is adhered to the sensor body such that the membrane system substantially covers the sensing region.

In an aspect of the first embodiment, the first material includes at least one material selected from the group consisting of plastics, metals, ceramics, and combinations thereof.

In an aspect of the first embodiment, the first material includes a plastic material.

In an aspect of the first embodiment, the plastic material includes a thermoset material.

In an aspect of the first embodiment, the thermoset material includes an epoxy.

In an aspect of the first embodiment, the plastic material includes a thermoplastic material.

In an aspect of the first embodiment, the sensor body further includes an insert formed from a second material, wherein the insert is situated within the sensor body or on the sensor body at a location substantially within the sensing region or around the sensing region.

In an aspect of the first embodiment, the second material includes a plastic material.

In an aspect of the first embodiment, the plastic material includes a thermoplastic material.

In an aspect of the first embodiment, the plastic material includes a thermoset material.

In an aspect of the first embodiment, the membrane system includes a plastic film.

In an aspect of the first embodiment, the membrane system includes a thermoplastic film or a thermoset film.

In an aspect of the first embodiment, the membrane is adhered to the body by application of heat.

In an aspect of the first embodiment, the membrane is adhered to the body by solvent welding.

In an aspect of the first embodiment, the membrane is adhered to the body by an adhesive.

In an aspect of the first embodiment, the membrane system is adhered to the body by application of pressure.

In an aspect of the first embodiment, the sensor body includes a substantially curved surface.

In an aspect of the first embodiment, the sensing region extends outward from a portion of the sensor body.

In an aspect of the first embodiment, the sensing region includes a convexly curved surface.

In an aspect of the first embodiment, the membrane system includes at least one component selected from the group consisting of a cell disruptive domain, a cell impermeable domain, a resistance domain, an enzyme domain, an interference domain, and an electrolyte domain.

In an aspect of the first embodiment, the sensing region includes a sensing mechanism selected from the group consisting of enzymatic, chemical, physical, optical, electrochemical, spectrophotometric, polarimetric, amperometric, calorimetric, and radiometric.

In an aspect of the first embodiment, the device further includes a disc adapted to adhere at least a periphery of the membrane system to the sensor body.

In an aspect of the first embodiment, the device further includes a ridge substantially surrounding a periphery of the membrane system when the membrane system is placed over the sensing region.

In an aspect of the first embodiment, the device further includes an inset portion within the sensor body, wherein the inset portion is configured to receive the membrane system.

In an aspect of the first embodiment, the device further includes a groove surrounding the sensing region.

In an aspect of the first embodiment, the membrane system is adhered at its periphery to the sensor body with sufficient strength to withstand in vivo cellular forces.

In a second embodiment, a method for manufacturing an analyte-measuring device including a sensing region for measuring the analyte is provided, the method including providing a membrane system; placing the membrane system on the analyte measuring device so as to cover the sensing region; and adhering at least a peripheral portion of the membrane system to the analyte measuring device such that analyte transport occurs only by diffusion through the membrane system.

In an aspect of the second embodiment, the adhering step includes adhering the membrane system to the device at a periphery of the membrane system, wherein a resulting bond between the device and the membrane system is sufficient strength to withstand in vivo cellular forces.

In an aspect of the second embodiment, the adhering step includes adhering using thermal energy.

In an aspect of the second embodiment, the thermal energy includes ultrasonic welding.

In an aspect of the second embodiment, the adhering step includes adhering using solvent welding.

In an aspect of the second embodiment, the adhering step includes applying an adhesive.

In an aspect of the second embodiment, the adhering step includes applying pressure.

In an aspect of the second embodiment, the adhering step includes applying a hot die over the membrane system.

In an aspect of the second embodiment, the adhering step includes attaching a disc to the device so as to secure the membrane system therebetween, wherein the disc is adapted to be placed over the membrane system and is configured to cover at least a periphery of the membrane system.

In an aspect of the second embodiment, the device includes a portion with a ridge configured to surround the membrane system, and wherein the adhering step molds the ridge over the membrane system.

In a third embodiment, an implantable glucose-measuring device is provided, including a sensor body including a thermoset material, wherein the sensor body includes a sensing region for measuring glucose; an insert including a thermoplastic material, wherein the insert is situated within the sensor body at a location substantially within the sensing region or surrounding the sensing region; and a membrane system permitting passage of the analyte at least partially therethrough, wherein the membrane system is adhered to the sensor body on the insert such that the membrane system substantially covers the sensing region.

In an aspect of the third embodiment, the membrane system is adhered to the insert by application of heat.

In an aspect of the third embodiment, the membrane system is adhered to the insert such that the periphery of the membrane system is sealed to the insert.

### Brief Description of the Drawings

Fig. 1A is a view of an unassembled analyte-measuring device, including a body with a membrane system to be adhered to the device body.

Fig. 1B is an assembled view of the analyte-measuring device of Fig. 1A, showing the body and the membrane system after adhesion.

Fig. 2A is a side schematic view of a membrane system in one embodiment, including a cell disruptive domain, a cell impermeable domain, a resistance domain, an enzyme domain, an interference domain, and an electrolyte domain.

Fig. 2B is a side schematic view of a membrane system in an alternative embodiment, including a biointerface membrane and a sensing membrane.

Fig. 2C is a side schematic view of a membrane system in another alternative embodiment, including a cell impermeable domain, a resistance domain, and an enzyme domain.

Fig. 3 is a flow chart that illustrates the process for manufacture of an analyte-measuring device with a membrane system in one embodiment.

Fig. 4A is a perspective view of an analyte-measuring device in one embodiment comprising a body with a plastic insert disposed therein surrounding and/or encompassing the sensing region.

Fig. 4B is a perspective view of the device of Fig. 4A, wherein the insert includes a fill material that surrounds the sensing mechanism.

Fig. 4C is a perspective view of the process of adhering a membrane system to the device of Fig. 4B in one embodiment.

Fig. 4D is a perspective view of the device of Fig. 4C, after the adhesion process.

Figs. 5A and 5B are perspective and side cross-sectional views of a membrane adhesion process in one embodiment.

Figs. 6A and 6B are perspective and side cross-sectional views of a membrane adhesion process in an alternative embodiment, wherein the membrane is sandwiched between the plastic insert and a circular donut or disc.

Figs. 7A and 7B are perspective and side cross-sectional views of a membrane adhesion process in another alternative embodiment, wherein the plastic insert includes a ridge substantially surrounding the periphery of the membrane system.

Figs. 8A and 8B are unassembled and assembled perspective views of one alternative embodiment of an analyte measuring device including an inset portion located thereon.

Figs. 9A and 9B are unassembled and assembled perspective views of another alternative embodiment of an analyte measuring device including a groove surrounding the sensing region.

Figs. 10A and 10B are unassembled and assembled perspective views of another alternative embodiment of an analyte measuring device, wherein an inner membrane and outer membrane are designed to slide over a smooth device surface.

Figs. 11A and 11B are unassembled and assembled perspective views of another alternative embodiment of an analyte measuring device, wherein a membrane attachment mechanism includes an insert that interlocks with a ring, which fits into the device body.

### Detailed Description of the Preferred Embodiment

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

### Definitions

In order to facilitate an understanding of the disclosed invention, a number of terms are defined below.

The term “thermoplastic,” as used herein, is a broad term and is used in its ordinary sense, including, but not limited to, materials that soften or melt when heated and harden when cooled. Thermoplastic polymers consist of long polymer molecules that are not linked to each other, namely, have no crosslinks. Some thermoplastics include polyethylene, polypropylene, polystyrene, polyester, polyvinyl chloride, acrylics, nylons, spandex-type polyurethanes, and cellulosics.

The term “thermoset,” as used herein, is a broad term and is used in its ordinary sense, including, but not limited to, materials that cannot be softened on heating. In thermosetting polymers, the polymer chains are joined (or crosslinked) by intermolecular bonding. Thermosets are usually supplied as partially polymerized or as monomer-polymer mixtures. Crosslinking is achieved during fabrication using chemicals, heat, or radiation; this process is called curing or vulcanization. Thermosets include, but are not limited to, phenolics, ureas, melamines, epoxies, polyesters, silicones, rubbers, acrylates, and polyurethanes.

The terms “membrane system” and “membrane” as used herein, are broad terms and are used in their ordinary sense, including, but not limited to, a membrane comprising one or more domains, layers, regions, or portions.

The term “domain” as used herein is a broad term and is used in its ordinary sense, including, without limitation, regions of the biocompatible membrane that can include layers, uniform or non-uniform gradients (for example, anisotropic), functional aspects of a material, or provided as portions of the membrane.

The term “hydrophile” and “hydrophilic” as used herein are broad terms and are used in their ordinary sense, including, without limitation, a chemical group that has a strong affinity for water. Representative hydrophilic groups include, but are not limited, to hydroxyl, amino, amido, imido, carboxyl, sulfonate, alkoxy, ionic, and other similar groups.

The term “hydrophobe” and “hydrophobic” as used herein are broad terms and are used in their ordinary sense, including, without limitation, a chemical group that does not readily absorb water, is adversely affected by water, or is insoluble in water.

The term “biointerface membrane” as used herein is a broad term and is used in its ordinary sense, including, without limitation, a permeable membrane that functions as a device-tissue interface comprised of one or more domains. In some embodiments, the biointerface membrane is composed of two domains. The first domain supports tissue ingrowth, interferes with barrier cell layer formation, and includes an open cell configuration having cavities and a solid portion. The second domain is impermeable to cells and cell processes (for example, macrophages). The biointerface membrane is made of biostable materials and can be constructed in layers, uniform or non-uniform gradients (for example, anisotropic), or in a uniform or non-uniform cavity size configuration.

The term “sensing membrane,” as used herein, is a broad term and is used in its ordinary sense, including, without limitation, a permeable or semi-permeable membrane that can be comprised of two or more domains and is typically constructed of materials of a few microns thickness or more, which are permeable to oxygen and may or may not be permeable to glucose. In one example, the sensing membrane comprises an enzyme, for example, immobilized glucose oxidase enzyme, which enables an electrochemical reaction to occur to measure a concentration of analyte.

The term “barrier cell layer” as used herein is a broad term and is used in its ordinary sense, including, without limitation, a cohesive monolayer of cells (for example, macrophages and foreign body giant cells) that substantially blocks the transport of molecules across the second domain and/or membrane.

The term “cellular attachment,” as used herein is a broad term and is used in its ordinary sense, including, without limitation, adhesion of cells and/or cell processes to a material at the molecular level, and/or attachment of cells and/or cell processes to micro- (or macro-) porous material surfaces. One example of a material used in the prior art that allows cellular attachment due to porous surfaces is the BIOPORE™ cell culture support marketed by Millipore (Bedford, MA).

The phrase “distal to” as used herein is a broad term and is used in its ordinary sense, including, without limitation, the spatial relationship between various elements in comparison to a particular point of reference. For example, some embodiments of a device include a biointerface membrane having a cell disruptive domain and a cell impermeable domain. If the sensor is deemed to be the point of reference and the cell disruptive domain is positioned farther from the sensor, then that domain is distal to the sensor.

The term “proximal to” as used herein is a broad term and is used in its ordinary sense, including, without limitation, the spatial relationship between various elements in comparison to a particular point of reference. For example, some embodiments of a device include a biointerface membrane having a cell disruptive domain and a cell impermeable domain. If the sensor is deemed to be the point of reference and the cell impermeable domain is positioned nearer to the sensor, then that domain is proximal to the sensor.

The term “cell processes” as used herein is a broad term and is used in its ordinary sense, including, without limitation, pseudopodia of a cell.

The term “solid portions” as used herein is a broad term and is used in its ordinary sense, including, without limitation, a solid material having a mechanical structure that demarcates the cavities, voids, or other non-solid portions.

The term “co-continuous” as used herein is a broad term and is used in its ordinary sense, including, without limitation, a solid portion wherein an unbroken curved line in three dimensions exists between any two points of the solid portion.

The term “biostable” as used herein is a broad term and is used in its ordinary sense, including, without limitation, materials that are relatively resistant to degradation by processes that are encountered in vivo.

The term “analyte” as used herein is a broad term and is used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-ß hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobinopathies, A,S,C,E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free ß-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I ; 17 alpha-hydroxyprogesterone; hypoxanthine phosphoribosyl transferase; immunoreactive trypsin; lactate; lead; lipoproteins ((a), B/A-1, ß); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky’s disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin; salts, sugar, protein, fat, vitamins and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

The terms “operably connected” and “operably linked” as used herein are broad terms and are used in their ordinary sense, including, without limitation, one or more components being linked to another component(s) in a manner that allows transmission of signals between the components. For example, one or more electrodes can be used to detect the amount of analyte in a sample and convert that information into a signal; the signal can then be transmitted to a circuit. In this case, the electrode is “operably linked” to the electronic circuitry.

The term “host” as used herein is a broad term and is used in its ordinary sense, including, without limitation, mammals, particularly humans.

The phrase “continuous (or continual) analyte sensing” as used herein is a broad term and is used in its ordinary sense, including, without limitation, the period in which monitoring of analyte concentration is continuously, continually, and or intermittently (regularly or irregularly) performed, for example, about every 5 to 10 minutes.

The term “sensing region” as used herein is a broad term and is used in its ordinary sense, including, without limitation, the region of a monitoring device responsible for the detection of a particular analyte. In one embodiment, the sensing region generally comprises a non-conductive body, a working electrode (anode), a reference electrode and a counter electrode (cathode) passing through and secured within the body forming an electrochemically reactive surface at one location on the body and an electronic connective means at another location on the body, and a multi-region membrane affixed to the body and covering the electrochemically reactive surface. The counter electrode has a greater electrochemically reactive surface area than the working electrode. During general operation of the sensor a biological sample (for example, blood or interstitial fluid) or a portion thereof contacts (directly or after passage through one or more membranes or domains) an enzyme (for example, glucose oxidase); the reaction of the biological sample (or portion thereof) results in the formation of reaction products that allow a determination of the analyte (for example, glucose) level in the biological sample. In some embodiments, the multi-region membrane further comprises an enzyme domain (for example, an enzyme layer), and an electrolyte phase (namely, a free-flowing liquid phase comprising an electrolyte-containing fluid described further below). However, the term is sufficiently broad so as to encompass a variety of sensing techniques, for example, enzymatic, chemical, physical, optical, electrochemical, spectrophotometric, polarimetric, amperometric, calorimetric, radiometric, and the like.

The terms “electrochemically reactive surface” and “electroactive surface” as used herein are broad terms and are used in their ordinary sense, including, without limitation, the surface of an electrode where an electrochemical reaction takes place. In the case of the working electrode, the hydrogen peroxide produced by the enzyme catalyzed reaction of the analyte being detected reacts creating a measurable electronic current (for example, detection of glucose analyte utilizing glucose oxidase produces H₂O₂ peroxide as a by product, H₂O₂ reacts with the surface of the working electrode producing two protons (2H⁺), two electrons (2e⁻) and one molecule of oxygen (O₂) which produces the electronic current being detected). In the case of the counter electrode, a reducible species, for example, O₂ is reduced at the electrode surface in order to balance the current being generated by the working electrode.

The term “oxygen antenna domain” as used herein is a broad term and is used in its ordinary sense, including, without limitation, a domain composed of a material that has higher oxygen solubility than aqueous media so that it concentrates oxygen from the biological fluid surrounding the biointerface membrane. The domain can then act as an oxygen reservoir during times of minimal oxygen need and has the capacity to provide on demand a higher oxygen gradient to facilitate oxygen transport across the membrane. This enhances function in the enzyme reaction domain and at the counter electrode surface when glucose conversion to hydrogen peroxide in the enzyme domain consumes oxygen from the surrounding domains. Thus, this ability of the oxygen antenna domain to apply a higher flux of oxygen to critical domains when needed improves overall sensor function.

The term “adhesive” as used herein is a broad term and is used in its ordinary sense, including, without limitation, a substance that enables adhesion between two elements. The substance can take a variety of forms, for example, a liquid adhesive or a joining material. The term adhesive is not limited to the type of material used in creating the adhesive joint between the two elements.

The term “adhere” and “attach” as used herein are a broad terms and are used in their ordinary sense, including, without limitation, to hold, bind, or stick, for example, by gluing, bonding, grasping, interpenetrating, or fusing.

The term “casting” as used herein is a broad term and is used in its ordinary sense, including, without limitation, a process where a fluid material is applied to a surface or surfaces and allowed to cure. The term is sufficiently broad so as to encompass a variety of coating techniques, for example, using a draw-down machine, dip coating, and the like.

### Overview

The present invention relates to the systems and methods associated with an analyte-measuring device that measures a concentration of analyte of interest or a substance indicative of the concentration or presence of the analyte. In some embodiments, the analyte-measuring device is a device that measures continuously, for example, a subcutaneous, transdermal, or intravascular device. In some embodiments, the device can analyze one or a plurality of intermittent blood samples. The analyte-measuring device can use any method of analyte-measurement, including enzymatic, chemical, physical, optical, electrochemical, spectrophotometric, polarimetric, calorimetric, amperometric, radiometric, or the like. The analyte-measuring device uses any known method, including invasive, minimally invasive, and non-invasive sensing techniques, to measure one or more analytes and to provide an output signal indicative of the concentration of the analyte or analytes of interest. The output signal is typically a raw signal that is used to provide a useful value of the analyte of interest to a user, such as a patient or doctor, using the device.

In general, analyte-measuring devices include a membrane system that functions to control the flux of a biological fluid therethrough and/or to protect sensitive regions of the device from contamination by the biological fluid, for example. Some conventional electrochemical enzyme-based analyte-measuring devices generally include a membrane system that controls the flux of the analyte being measured, protects the electrodes from contamination of the biological fluid, and/or provides an enzyme that catalyzes the reaction of the analyte with a co-factor, for example. See, e.g., co-pending U.S. Patent Application 10/838,912, filed May 3, 2004 entitled “IMPLANTABLE ANALYTE SENSOR,” which is incorporated herein by reference in its entirety.

Conventionally, membrane systems are attached to analyte-measuring devices using a variety of methods which can have various drawbacks. For example, the above-cited U.S. Patent Application teaches a raised sensing region around which the membrane is attached via a clip in a groove. In certain designs, this membrane attachment method can utilize a significant amount of physical space, which can limit efforts to reduce the size of the sensor. While not wishing to be bound by any particular theory, it is believed that design optimization (for example, reduction of size, mass, and/or profile) of the implantable analyte-measuring device enables a more discrete and secure implantation than a larger or higher profile device. Such design optimization is also believed to reduce macro-motion of the device induced by the patient and micro-motion caused by movement of the device within the subcutaneous pocket, thereby improving device performance.

Depending upon the method, attachment of the membrane system to the device can result in problems with the maintenance of the seal of the membrane system when the device is implanted. For example, the seal at the edges of the membrane system are preferably strong enough to resist the forces associated with cellular invasion in vivo and additionally preferably ensure that enzymes or other molecules that can invoke a xenogeneic response in vivo do not have a pathway to leak through the edges, such that transport of the analyte occurs via diffusion through the membrane system. Problems can sometimes be encountered in attaching a membrane to the device body due to the difficulty in attaching dissimilar materials without depending upon mechanical attachment.

Accordingly, the preferred embodiments provide systems and methods for attaching a membrane system to an analyte-measuring device, wherein the systems and methods can include: 1) efficient utilization of device volume; 2) overall reduction of device size; 3) a substantially damage-free membrane attachment process; 4) ease and cost-effectiveness of testing membranes on the device; 5) sealed edges such that biological fluid cannot grow under the membrane edges; and/or 6) sealed edges such that the enzyme does not invoke a xenogeneic response with the biological fluid.

### Description

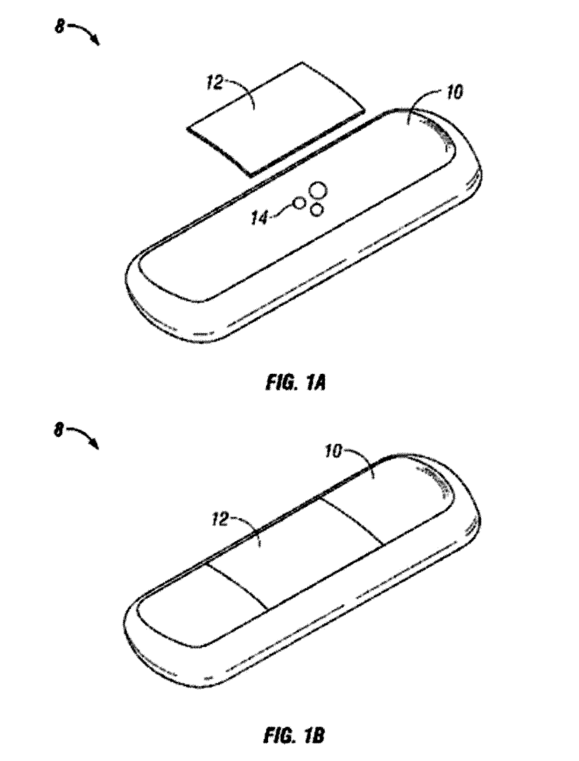

Figs. 1A and 1B are perspective views of an implantable analyte-measuring device in one embodiment. Fig. 1A is a perspective view of an unassembled analyte-measuring device 8, including a body 10 with a membrane system 12 to be adhered over the sensing region 14, which is an electrode system in the illustrated embodiment. Fig. 1B is an assembled view of the analyte-measuring device 8 of Fig. 1A, showing a body 10 and the membrane system 12 after attachment.

The body 10 of the device 8 can be formed from a variety of materials, including metals, ceramics, plastics, or composites thereof. In one embodiment, the device is formed from thermoset molded around the device electronics. Co-pending U.S. Patent Application No. 10/646,333, entitled, “OPTIMIZED DEVICE GEOMETRY FOR AN IMPLANTABLE GLUCOSE DEVICE” discloses suitable configurations for the body, and is incorporated by reference in its entirety.

In one preferred embodiment, the device 8 is an electrochemical enzyme-based device, wherein the sensing region 14 includes an electrode system (for example, a platinum working electrode, a platinum counter electrode, and a silver/silver chloride reference electrode), which is described in more detail with reference to U.S. Patent Application 09/916,711, entitled “SENSOR HEAD FOR USE WITH IMPLANTABLE DEVICES,” which is incorporated herein by reference in its entirety. However, a variety of electrode materials and configurations can be used with the implantable analyte-measuring device of the preferred embodiments. The top ends of the electrodes are in contact with an electrolyte phase (not shown), which is a free-flowing fluid phase disposed between the membrane system 12 and the electrode system. In this embodiment, the counter electrode is provided to balance the current generated by the species being measured at the working electrode. In the case of a glucose oxidase based analyte-measuring device, the species being measured at the working electrode is H₂O₂. Glucose oxidase catalyzes the conversion of oxygen and glucose to hydrogen peroxide and gluconate according to the following reaction:

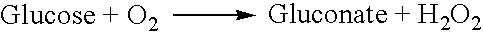

The change in H₂O₂ can be monitored to determine glucose concentration because for each glucose molecule metabolized, there is a proportional change in the product H₂O₂. Oxidation of H₂O₂ by the working electrode is balanced by reduction of ambient oxygen, enzyme generated H₂O₂, or other reducible species at the counter electrode. The H₂O₂ produced from the glucose oxidase reaction further reacts at the surface of the working electrode and produces two protons (2H⁺), two electrons (2e⁻), and one oxygen molecule (O₂).

In this embodiment, a potentiostat is employed to monitor the electrochemical reaction at the electroactive surface(s). The potentiostat applies a constant potential to the working and reference electrodes to determine a current value. The current that is produced at the working electrode (and flows through the circuitry to the counter electrode) is substantially proportional to the amount of H₂O₂ that diffuses to the working electrode. Accordingly, a raw signal can be produced that is representative of the concentration of glucose in the user’s body, and therefore can be utilized to estimate a meaningful glucose value.

Although the preferred embodiments describe and illustrate one type of an electrochemical analyte-measuring device, it should be appreciated that the associated systems and methods for attaching the membrane system to the device can be implemented with a wide variety of known analyte-measuring devices, including chemical, physical, optical, electrochemical, spectrophotometric, polarimetric, amperometric, calorimetric, radiometric, or the like. Some analyte-measuring devices that can benefit from the systems and methods of the preferred embodiments include U.S. Patent No. 5,711,861 to Ward et al., U.S. Patent No. 6,642,015 to Vachon et al., U.S. Patent No. 6,654,625 to Say et al., U.S. Patent No. 6,514,718 to Heller, U.S. Patent No. 6,465,066 to Essenpreis et al., U.S. Patent No. 6,214,185 to Offenbacher et al., U.S. Patent No. 5,310,469 to Cunningham et al., and U.S. Patent No. 5,683,562 to Shaffer et al., for example. All of the above patents are incorporated in their entirety herein by reference and are not inclusive of all applicable analyte-measuring devices; in general, it should be understood that the disclosed embodiments are applicable to a variety of analyte-measuring device configurations.

### Membrane System

In general, the membrane system 12 can include any membrane configuration suitable for use with any analyte-measuring device. In the illustrated embodiments, the membrane system includes a plurality of domains, all or some of which can be adhered to the analyte-measuring device 8 via the systems and methods described herein.

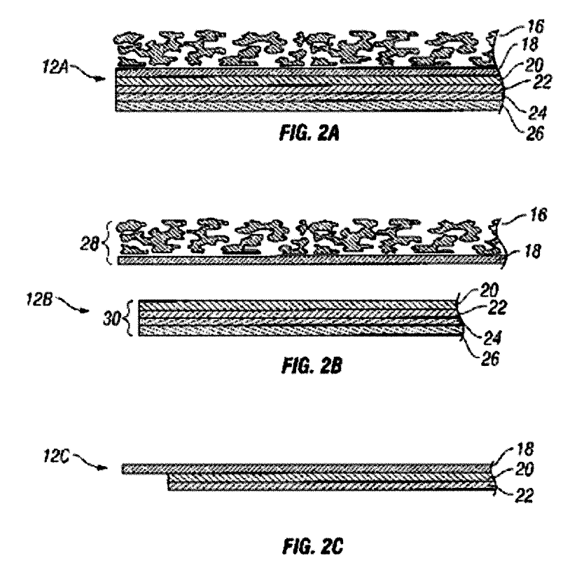

Fig. 1B illustrates an analyte-measuring device in one embodiment including a membrane system 12 adhered over the sensing region, wherein the membrane system includes one or more of the following domains: a cell disruptive domain, a cell impermeable domain, a resistance domain, an enzyme domain, an interference domain, and an electrolyte domain, such as described in more detail with reference to Figs. 2A to 2C. However, it is understood that the membrane system 12 can be modified for use in other devices, by including only one or more of the domains, or additional domains not recited above. For example, the interference domain can be removed when other methods for removing interferants are utilized. As another example, an “oxygen antenna domain” composed of a material that has higher oxygen solubility than aqueous media so that it concentrates oxygen from the biological fluid surrounding the biointerface membrane can be added. The oxygen antenna domain can then act as an oxygen source during times of minimal oxygen availability and has the capacity to provide on demand a higher rate of oxygen delivery to facilitate oxygen transport to the membrane. This enhances function in the enzyme reaction domain and at the counter electrode surface when glucose conversion to hydrogen peroxide in the enzyme domain consumes oxygen from the surrounding domains. Thus, this ability of the oxygen antenna domain to apply a higher flux of oxygen to critical domains when needed improves overall sensor function. Reference is made to Figs. 2A to 2C, which illustrate domains of a membrane system in some preferred embodiments.

### Cell disruptive domain

The cell disruptive domain 16 comprises a solid portion and a plurality of interconnected three-dimensional cavities formed therein. In one embodiment, the cavities have sufficient size and structure to allow invasive cells, such as fibroblasts, fibrous matrix, and blood vessels to completely enter into the apertures that define the entryway into each cavity, and to pass through the interconnected cavities toward the device. The cavities comprise an architecture that encourages the ingrowth of vascular tissue in vivo and reduces or prevents barrier cell layer formation. Because of the vascularization within the cavities, solutes (e.g., oxygen, glucose and other analytes) can pass through the first domain with relative ease and/or the diffusion distance (i.e., distance that the glucose diffuses) can be reduced. U.S. Patent No. 5,741,330, U.S. Patent Application No. 10/647,065, and U.S. Provisional Patent Application No. 60/544,722, all of which are incorporated herein by reference in their entirety, describe porous membranes that can be used in the preferred embodiments. Additionally, a variety of known porous biointerface materials suitable for implantable devices can be used as is appreciated by one skilled in the art. It is noted that the cell disruptive domain can be useful in long-term implantable analyte-measuring devices; however, this domain can be eliminated for non-implantable or short-term implantable analyte-measuring devices, for example.

### Cell impermeable domain

The cell impermeable domain 18 is impermeable to cells and cell processes and protects the underlying membrane and device from biological contamination. In some embodiments, the cell impermeable domain can be resistant to cellular attachment and thus provides another mechanism for resisting barrier cell layer formation; because the cell impermeable domain 18 is resistant to cellular attachment and barrier cell layer formation, the transport of solutes such as described above can also pass through with relative ease without blockage by barrier cells as seen in the prior art.

Generally, the materials that are preferred to form this domain, for example, polycarbonate-based polyurethanes, silicones, and other such materials described herein, are resistant to the effects of these oxidative species and have thus been termed “biodurable”. Additionally, the materials are substantially hydrophilic so as to permit the transport of selected analytes therethrough. See, e.g., U.S. Patent Application No. 09/916386, filed July 27, 2001, and entitled “MEMBRANE FOR USE WITH IMPLANTABLE DEVICES” and U.S. Patent Application No. 10/647,065, filed August 22, 2003, and entitled, “POROUS MEMBRANES FOR USE WITH IMPLANTABLE DEVICES,” which are incorporated herein by reference in their entirety.

### Resistance domain

The resistance domain 20 includes a semipermeable membrane that controls the flux of analytes of interest (for example, glucose and oxygen) to the underlying enzyme domain 22. As a result, the upper limit of linearity of an analyte measurement can be extended to a much higher value than what can be achieved without the resistance domain. In one embodiment of a glucose-measuring device, the resistance domain 20 exhibits an oxygen-to-glucose permeability ratio of approximately 200:1. As a result, one-dimensional reactant diffusion is adequate to provide excess oxygen at all reasonable glucose and oxygen concentrations found in the subcutaneous matrix (See Rhodes et al., Anal. Chem., 66:1520-1529 (1994)).

In some alternative embodiments, a lower ratio of oxygen-to-glucose can be sufficient to provide excess oxygen by using an oxygen antenna domain (for example, a silicone or fluorocarbon based material or domain) to enhance the supply/transport of oxygen to the enzyme domain. In other words, if more oxygen is supplied to the enzyme, then more glucose can also be supplied to the enzyme without the rate of this reaction being limited by a lack of glucose. In some alternative embodiments, the resistance domain is formed from a silicone composition, such as described in copending U.S. Application No. 10/685,636 filed October 28, 2003, and entitled, “SILICONE COMPOSITION FOR BIOCOMPATIBLE MEMBRANE,” which is incorporated herein by reference in its entirety.

In one preferred embodiment, the resistance layer includes a homogenous polyurethane membrane with both hydrophilic and hydrophobic regions to control the diffusion of glucose and oxygen to an analyte-measuring device, the membrane being fabricated easily and reproducibly from commercially available materials. In preferred embodiments, the thickness of the resistance domain is from about 10 microns or less to about 200 microns or more.

### Enzyme domain

In the preferred embodiments, the enzyme domain 22 provides a catalyst to catalyze the reaction of the analyte and its co-reactant, as described in greater detail above. In preferred embodiments, the enzyme domain includes glucose oxidase. However other oxidases, for example, galactose oxidase or uricase, can be used.

For example, enzyme-based electrochemical glucose-measuring device performance at least partially depends on a response that is neither limited by enzyme activity nor cofactor concentration. Because enzymes, including glucose oxidase, are subject to deactivation as a function of ambient conditions, this behavior needs to be accounted for in constructing analyte-measuring devices. Preferably, the domain is constructed of aqueous dispersions of colloidal polyurethane polymers including the enzyme. However, some alternative embodiments construct the enzyme domain from an oxygen antenna material, for example, silicone or fluorocarbons, in order to provide a supply of excess oxygen during transient ischemia. Preferably, the enzyme is immobilized within the domain, as is appreciated by one skilled in the art.

### Interference domain

Interferants are molecules or other species that are electro-reduced or electro-oxidized at the electrochemically reactive surfaces, either directly or via an electron transfer agent, to produce a false signal. In one embodiment, the interference domain 24 prevents the penetration of one or more interferants (for example, ureate, ascorbate, or acetaminophen) into the electrolyte phase around the electrochemically reactive surfaces. Preferably, this type of interference domain is much less permeable to one or more of the interferants than to the analyte.

In one embodiment, the interference domain 24 can include ionic components incorporated into a polymeric matrix to reduce the permeability of the interference domain to ionic interferants having the same charge as the ionic components. In another embodiment, the interference domain 24 includes a catalyst (for example, peroxidase) for catalyzing a reaction that removes interferants. U.S. Patent 6,413,396 andU.S. Patent 6,565,509 disclose methods and materials for eliminating interfering species. However, in the preferred embodiments any suitable method or material can be employed.

In one embodiment, the interference domain 24 includes a thin membrane that is designed to limit diffusion of species, e.g., those greater than 34 g/mol in molecular weight, for example. The interference domain permits analytes and other substances (for example, hydrogen peroxide) that are to be measured by the electrodes to pass through, while preventing passage of other substances, such as potentially interfering substances. In one embodiment, the interference domain 24 is constructed of polyurethane.

### Electrolyte domain

In some preferred embodiments, an electrolyte domain 26 is provided to ensure an electrochemical reaction occurs at the electroactive surfaces. Preferably, the electrolyte domain includes a semipermeable coating that maintains hydrophilicity at the electrochemically reactive surfaces of the sensor interface. The electrolyte domain enhances the stability of the interference domain 26 by protecting and supporting the material that makes up the interference domain. The electrolyte domain also assists in stabilizing the operation of the sensor by overcoming electrode start-up problems and drifting problems caused by inadequate electrolyte. The buffered electrolyte solution contained in the electrolyte domain also protects against pH-mediated damage that can result from the formation of a large pH gradient between the substantially hydrophobic interference domain and the electrodes due to the electrochemical activity of the electrodes. In one embodiment, the electrolyte domain 26 includes a flexible, water-swellable, substantially solid gel-like film.

The above-described domains are exemplary and are not meant to be limiting to the following description, for example, their systems and methods are designed for the exemplary enzyme-based electrochemical sensor embodiment.

### Exemplary Membrane Configurations

The systems and methods of the preferred embodiments can be applied to a variety of membrane configurations including one or more of the above-described domains. Figs. 2A to 2C illustrate three exemplary membrane systems that can be used with an analyte-measuring device.

Fig. 2A is a side schematic view of a membrane system 12a in one embodiment, including a cell disruptive domain 16, a cell impermeable domain 18, a resistance domain 20, an enzyme domain 22, an interference domain 24, and an electrolyte domain 26. In this embodiment, the domains can be formed as one system and together adhered to the analyte-measuring device, for example.

Fig. 2B is a side schematic view of a membrane system 12b in another embodiment, including: 1) a cell disruptive domain 16 and a cell impermeable domain 18, hereinafter referred to as the biointerface membrane 28, which can be formed, placed, or attached together; and 2) a resistance domain 20, an enzyme domain 22, an interference domain 24, and an electrolyte domain 26, hereinafter referred to as the sensing membrane 30, which can be formed or attached together. The term “biointerface membrane” generally refers to the one or more membrane domains that are adapted to contact host tissue when implanted. The term “sensing membrane” generally refers to the underlying membrane domains proximal to the sensing region of the device and can provide functionality that aids or protects the sensing mechanism. The terms “sensing membrane” and “biointerface membrane” are not limited to the configuration of biointerface and sensing membranes of this embodiment, as is appreciated by one skilled in the art.

Advantages of forming the membrane system in more than one piece, for example as in the distinct sensing membrane 30 and biointerface membrane 28 of Fig. 2B, include unique manufacturability, attachment considerations, and/or other design considerations. For example, it can be preferred that the sensing membrane 30 includes an edge sealing step that ensures no leakage of the enzyme therefrom or traversing of uncontrolled analyte into the edges thereof. As another example, it can be preferred that the sensing membrane 30 be adhered substantially entirely across the surface of the membrane to the device in order to maintain tautness when hydrated. As yet another example, it can be preferred that the biointerface membrane 28 be adhered only at its periphery to protect the central portion of the membrane from damage that can result from the attachment process.

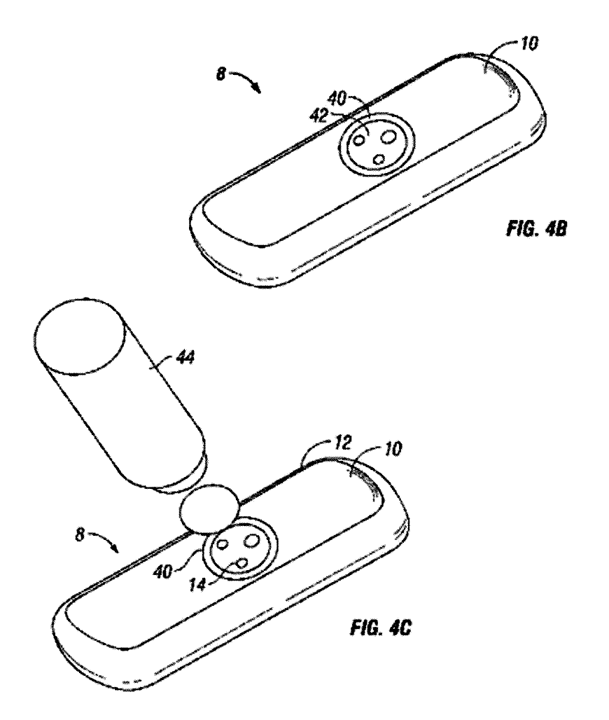

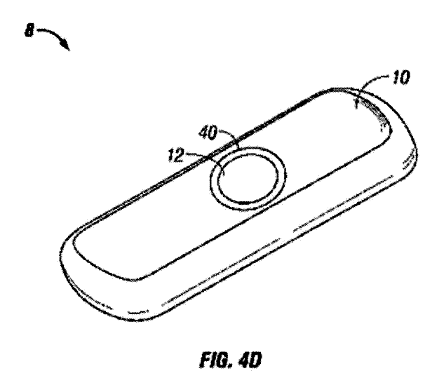

Fig. 2C is a side schematic view of a membrane system 12c in yet another embodiment, including: a cell impermeable domain 18, a resistance domain 20, and an enzyme domain 22. In the illustrated embodiment, the cell impermeable domain 18 extends peripherally farther than the other two domains; one advantage of this configuration includes the ability to adhere only one of the domains to the body, while effectively sealing all domains from the biological environment. It is noted that some analyte-measuring devices may not include a cell disruptive domain, for example those designed for a short implant time, or those with other design considerations. It is further noted that some analyte-measuring devices may not include an interference domain, for example devices for use when substantially no interferants exist, or devices for use when interferants are excluded or eliminated using other (for example, electrochemical) methods. It is further noted that some analyte-measuring devices may not include an electrolyte domain, for example devices wherein the sensing mechanism does not use electrochemical techniques, or devices wherein the electrolyte function is provided in another manner (for example, applied as a liquid film as described in more detail with reference to Fig. 4C).

It is noted that the membrane system 12 can be divided along any of the domains 16, 18, 20, 22, 24, and 26 when separate manufacturing and/or attachment techniques or considerations can be advantageous. The following description of membrane attachment encompasses any membrane system that can be used on an analyte-measuring device and that allows the transport of at least one analyte therethrough.

### Membrane Attachment

In order to minimize the amount of space required by the attachment method while maximizing adhesion and longevity of the membrane on the device, the preferred embodiments can provide a method for manufacture, include adhering of a membrane system to an analyte-measuring device that enables: 1) efficient utilization of device volume; 2) overall reduction of device size; 3) a substantially damage-free membrane attachment process; 4) ease and cost-effectiveness of testing membranes (for example, pre-attached) on the device; 5) sealed edges such that biological substances (namely, cells) cannot grow under the membrane edges; and/or 6) sealed edges such that the enzyme does not invoke a xenogeneic response with the biological fluid.

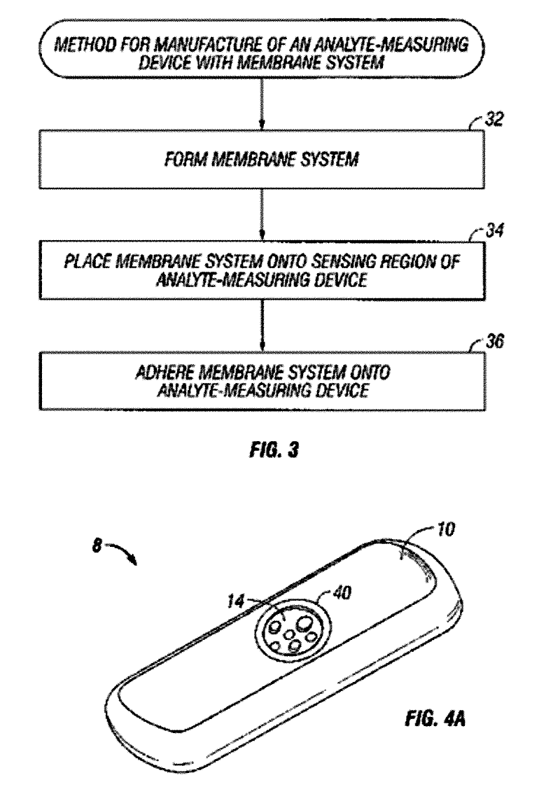

Fig. 3 is a flow chart that illustrates a process for manufacture of an analyte-measuring device with a membrane system. At block 32, a membrane system 12 is formed using techniques known to those skilled in the art. For example, the membrane system can be serially cast or cast on a continuous web machine to produce a membrane system 12 with a configuration suitable for an analyte-measuring device, such as described in more detail with reference to Figs. 2A to 2C. Co-pending U.S. Patent Application No. 10/838,912, filed May 3, 2004, and entitled “IMPLANTABLE ANALYTE SENSOR,” which is incorporated herein by reference in its entirety, describes one method for manufacturing a membrane system as described herein.

At block 34, the membrane system 12 is placed over the sensing region 14 of analyte-measuring device. Some or all of the membrane system is placed over the sensing region (for example, the electrode system in an electrochemical-based device). In some embodiments, an adhesive is applied to the sensing region and/or the portion of the membrane system to be placed over the sensing region, hereinafter referred to as “primer.” The purpose of this primer is to ensure complete contact of the membrane with the sensing region in the assembled analyte-measuring device. Complete contact of the membrane with the sensing region using a primer minimizes the risk of wrinkling of the membrane or bubble formation between the membrane and sensing region during or after the subsequent adhesion process 36. However, in some embodiments, the primer may not be required.

In one embodiment, the primer is a liquid form of the electrolyte domain 26 applied to the sensing region of the device prior to placement of a substantially non-hydrated membrane system in order to ensure adhesion of the membrane system to the device during and after adhesion and hydration of the device. In some embodiments, however, even when a fully hydrated membrane is placed on the device prior to the subsequent adhesion process 36, primer can be beneficial for maintaining substantial tautness such that the membrane can be attached without incurring wrinkles or bubbles during subsequent processing.

At block 36, the membrane system is attached or adhered onto the analyte-measuring device. As discussed in more detail in the Overview section above, it can be advantageous to seal the edges of the membrane such that biological substances (namely, cells) cannot grow under the membrane edges and such that the enzyme or other such foreign substances from the membrane do not invoke a xenogeneic response with the biological fluid.

In one embodiment, all domains of the membrane extend to the same edge, such as is illustrated in Fig. 2A. In some embodiments, attachment or adhesion is preferably performed at the outermost periphery of the membrane system to ensure complete sealing with no leakage. In alternative embodiments, such as are illustrated in Figs. 2B and 2C, at least one domain 18, preferably an upper portion, extends to an edge that is outside the periphery of the other edges of domains 20, 22, and 26. In these embodiments, the adhesion process is preferably applied only to the upper domain 18 that extends external to the other domains; in this way, the adhesion process affects only part of the membrane system, while sealing all the domains from contact with the biological fluid. In yet other alternative embodiments, it is not required that the edges be sealed.

In one embodiment, the membrane system 12 is thermally adhered to the device body 10, which is described in more detail with reference to Fig. 4C. Thermal adhesion generally refers to an adhesive joint formed by heat that causes a melt of the various materials, forming a strong attachment between the membrane system and the device. Alternatively, solvent welding or liquid adhesives can be used, which are described in more detail elsewhere herein. As yet another alternative, the membrane system can be adhered by pressure to the device body, as is appreciated by one skilled in the art.

In general, membranes for use with analyte-measuring devices are substantially plastic films. It is noted, for example, that membranes used with amperometric analyte-measuring devices can be thermoplastic, hydrophilic membranes that allow the transport of analytes therethrough. As another example, membranes used with spectrophotometric analyte-measuring devices can be hydrophobic in nature. Additionally, analyte-measuring devices are generally formed from plastic, ceramic, metal, or some combination thereof. Unfortunately, when the membrane material is not substantially similar to the device material to which it is being adhered, a strong adhesive joint can sometimes be difficult to achieve. For example, hydrophilic, thermoplastic membranes are difficult to bond to many thermoset materials at temperatures that are suitable for these manufacturing processes, due to their dissimilarity; in this case, it can be advantageous to provide a portion of the device formed from a thermoplastic material that provides a surface optimized for attaching the thermoplastic membrane system to the device, wherein the materials are designed to ensure a strong adhesive joint in the region of attachment. Figs. 4A to 4D illustrate one embodiment that provides a plastic insert for these purposes. However, other configurations and materials incorporated into the device are within the scope of the preferred embodiments.

A variety of thermal attaching techniques can be used with the preferred embodiments, including hot air gun, hot knife welding, hot plate welding, dielectric welding, high frequency welding, hot-gas welding, induction (impulse) welding, laser welding, sonic welding, ultrasonic welding, or the like. Welding processes are particularly advantageous, as they have been shown to consistently and reliably seal the membrane to the device body with reduced risk of leakage or delamination. For example, laser welding is known to produce a high quality weld seam at processing speeds that result in outstanding productivity and efficiency, leading to reduced operating costs, increased speed of device manufacture, and the capability of processing at high powers using a single source.

In some alternative embodiments, the membrane system can be adhered to the device using solvent welding. Solvent welding is a process wherein a solvent is applied which can temporarily swell the polymer at room temperature. When this occurs, the polymer chains are free to move in the liquid and can entangle with other dissolved chains in the other component. Given sufficient time, the solvent will permeate through the polymer and out into the environment, so that the chains lose their mobility. This leaves a solid mass of entangled polymer chains, which constitutes a solvent weld, also referred to herein as an adhesive joint. In some cases, heat can be applied to raise the temperature of the polymer above the transition temperature. Solvent welding can be advantageous in conditions where it can be advantageous to weld with minimal heat, for example.

In some additional alternative embodiments, the membrane system can be adhered to the device using an adhesive, such as a liquid or non-liquid adhesive. Examples of liquid adhesives include silicone, epoxy, and the like. Examples of non-liquid adhesives include joining materials, such as a polyurethane membrane, and the like. In these alternative embodiments the adhesive is applied to the membrane system in a manner such that the adhesive surrounds the sensing region upon application. For example, the liquid adhesive can be applied in a ring-like fashion around a region that surrounds the electrode system; however the shape of the adhesive application can be varied as desired. In another implementation, a fixture can be formed that allows the adhesive to be applied conforming to the configuration of each individual electrode. Other adhesive attachments are also possible. Because a liquid adhesive preferably does not seep onto the electrode surfaces, some embodiments can provide an inset or groove formed in the body around each (or collective) electrode(s) to direct the flow of the adhesive therein. Epoxy can be an advantageous adhesive in some embodiments wherein the device body is formed from epoxy, as it is homogeneous and is known to be biocompatible.

Figs. 4A to 4D are perspective views that illustrate steps in membrane adhesion of an analyte-measuring device in one embodiment. In the preferred embodiments, the device body, or a portion thereof, is preferably formed from a material that is substantially similar to the membrane system to enable strong adhesion therebetween. In some embodiments, the entire device body is formed from a material that is similar to the membrane system; while in other embodiments, only a portion (hereinafter referred to as an insert) is formed from a similar material. The embodiment illustrated below provides one example of how an insert can be disposed into the device body. However, numerous alternative configurations are within the scope of the preferred embodiments. It is noted that in embodiments wherein the device body is sufficiently similar to the membrane system to enable a strong adhesive joint therebetween, a separate insert material is not required.

Fig. 4A is a perspective view of an analyte-measuring device 8 comprising a body 10 with a plastic insert 40 disposed therein surrounding and/or encompassing the sensing region 14. In some embodiments, plastic insert 40 can be molded into the body 10, for example, when the body is formed from a molded material, such as is described in co-pending U.S. Patent Application 10/838,912 filed May 3, 2004, and entitled “IMPLANTABLE ANALYTE SENSOR,” which is incorporated herein by reference in its entirety. In some alternative embodiments, the insert is snap-fit, press-fit, adhered, or otherwise securely disposed within the body 10 of the device as is appreciated by one skilled in the art. In some embodiments, the insert 40 is raised from the surface of the device body 10. The material that forms the insert 40 can comprise any suitable plastic material, for example, polyethylene, polypropylene, polystyrene, polyester, polyvinyl chloride, acrylics, nylons, polyurethanes, cellulosics, acrylates, or the like. In one exemplary embodiment, the insert is formed from Carbothane® (available from Carboline Co., St. Louis, MO), which is a thermoplastic material suitable for forming an adhesive joint with a thermoplastic film using thermal energy, for example. In another exemplary embodiment, the insert is formed from an acrylate, which is a thermoset material suitable for forming an adhesive joint with a thermoset film using UV irradiation techniques. In general, the body or insert material can be formed from any plastic suitable for forming a strong adhesive joint with a membrane system of an analyte-measuring device, namely a material that is sufficiently similar to the membrane system to enable the strong adhesion such that transport of the analyte occurs only through diffusion of the membrane system 12. When desired, the insert 40 can be formed in any shape or dimension suitable for at least the adhesion process and can encompass a relatively small or substantial portion of the device. The illustrated implementation is in no way limiting to the configuration of the plastic “insert” or “portion” described herein.

In the exemplary embodiment of Fig. 4A, the sensing region 14 includes a three electrode system, which is operably connected to electronics housed within the body 10. In this embodiment, the body is preferably designed so as to minimize moisture penetration into the interior of the device (for example, to the electronics). Because a tight interface is formed between the electrodes and a thermoset material that is stronger than between the electrodes and a thermoplastic material, the body can be designed such that a thermoset material can be molded into the thermoplastic insert so as to minimize moisture penetration at the electrodes.

Fig. 4B is a perspective view of the body 10, wherein the plastic insert 40 is imbedded within the device body and filled with a fill material 42 that surrounds the sensing mechanism, such as described para supra. However, it is appreciated by one skilled in the art that this configuration is dependent upon the type of sensing mechanism, material combinations, methods of manufacture, or other design features of a particular analyte-measuring device, and can be varied as desired. It is noted that some embodiments include an insert material that fully encompasses the sensing mechanism, while other embodiments include an insert material exposed only in the region of the adhesive joint of the device, for example. In one alternative embodiment, the insert 40 is insert-molded as a subassembly and then formed into the device body 10. Accordingly, the filling step described herein is considered optional and its use can depend upon the device configuration.

Fig. 4C is a perspective view of the process of thermally attaching a membrane 12 to an analyte-measuring device 8 in one embodiment. In one embodiment, the insert 40 comprises a thermoplastic material, such as described in more detail above, and the membrane 12 comprises a substantially hydrophilic, thermoplastic film, such as described in more detail above.

Prior to the thermal adhesion process 36, it can be advantageous to provide a primer adhesive, herein referred to as “primer”, in order to ensure adhesion of the membrane system 12 to the device 8 during and after the adhesion process 36. For example, when a non-hydrated membrane 12 is adhered to a device 8, and then subsequently hydrated after the adhesion process 36, it can be susceptible to bubbling or wrinkling after hydration. Therefore, it can be advantageous to provide a primer step, wherein a layer of adhesive is applied to the membrane and/or sensing region in order to overcome the pressures and stresses incurred by the membrane 12 during and after the adhesion process 36, and in order to ensure full contact of the membrane 12 with the sensing region 14 over time. In one embodiment, the adhesive used in the primer step is a liquid form of the electrolyte domain hydrogel; one skilled in the art appreciates however other alternative materials are also possible. The primer step, however, is optional and may not be advantageous or desirable in all circumstances.

One additional advantage of the primer step includes the ability to do manufacturing testing, or the like, with the membrane system 12 over the device 8, prior to the subsequent adhesion process 36. For example, thermal adhesion onto a thermoplastic material typically produces surface modification of the thermoplastic material. Therefore, if a membrane system was determined to be faulty after thermal adhesion, some damage to the body or insert can be incurred by the device, although the device can still be re-workable with a new membrane system. However, an adhesive applied during the primer step is easily removable and therefore enables easy testing and rework of the device prior to the subsequent adhesion process 36.

In this embodiment, after optionally applying the primer to the membrane 12 and/or sensing region 14, a hot die 44 is pressed down over the membrane 12 and insert 40 to form an adhesive joint therebetween. In this way, a sufficiently strong adhesive joint between the membrane and the analyte-measuring device is formed, such that biological fluid cannot infiltrate or cells cannot grow under the membrane edges and/or the enzyme does not invoke a xenogeneic response with the biological fluid. All of the above discussion referring to adhesion to the insert is applicable to adhesion to the body in embodiments that do not include a separate insert, as discussed in more detail above.

Fig. 4D is a perspective view of the analyte-measuring device 8, after the adhesion process 36. In general, the membrane attachment of the preferred embodiments provides systems and methods for efficient utilization of the device volume, thereby enabling an overall reduction of device size. As described above, design optimization (for example, reduction of size, mass, and/or profile) of the implantable analyte-measuring device is believed to enable a more discrete and secure implantation than a larger device, and is believed to reduce macro-motion of the device induced by the patient and micro-motion caused by movement of the device within the subcutaneous pocket, and thereby improve device performance. In one preferred embodiment, an analyte-measuring device such as described in the preferred embodiments was designed and built with a length of about 1 inch, a width of about 0.44 inches, and a height of about 0.15 inches. While not wishing to be bound by theory, it is believed that an analyte-measuring device with these dimensions is less susceptible to motion artifact, requires a decreased invasiveness of implantation, and provides overall improved patient comfort and device performance, as compared to a larger or higher profile device, for example.

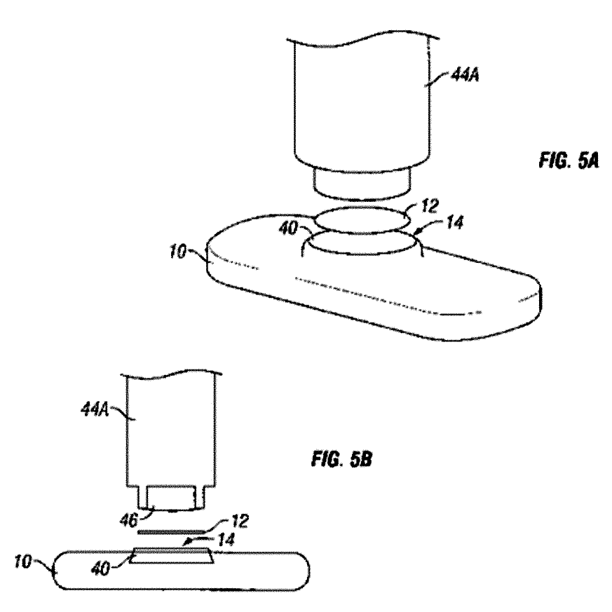

Figs. 5A to 7B are perspective and side cross-sectional views that illustrate various systems and methods for the thermal adhesion process 36 of the membrane 12 to a device body 10. Although a few exemplary embodiments are shown, they are not meant to be limiting to the preferred embodiments.

Figs. 5A and 5B are perspective and side cross-sectional views of the membrane adhesion process such as described with reference to Figs. 4A to 4D. This illustration exemplifies one alternative embodiment, wherein the sensing region 14 is located within an insert 40, one or both of which can be raised from the surface of the body 10 in certain embodiments, and wherein a hot die 44a includes an inset 46 to accommodate the raised sensing region 14 and such that the hot die does not touch the central portion of the membrane system 12 during the adhesion process. The raised configuration can be advantageous in that it is believed to provide improved tension or tautness of the membrane 12 over the sensing region 14 to decrease tendency of the membrane 12 to bubble or wrinkle, thereby providing a smoother, more consistent membrane attachment. In some embodiments, the raised sensing region 14 comprises a smooth, convexly curved surface, for example, to further decrease tendency of the membrane 12 to bubble or wrinkle, as described above. Additionally, it is advantageous that the sensing region be located at an apex of the device body. Employing a sensing region at the apex can optimize tissue healing at the device-tissue interface when the device is implanted in soft tissue, such as is described in detail with reference to co-pending U.S. Patent Application No. 10/646,333, entitled “OPTIMIZED SENSOR GEOMETRY FOR AN IMPLANTABLE GLUCOSE SENSOR,” which is incorporated herein by reference in its entirety.

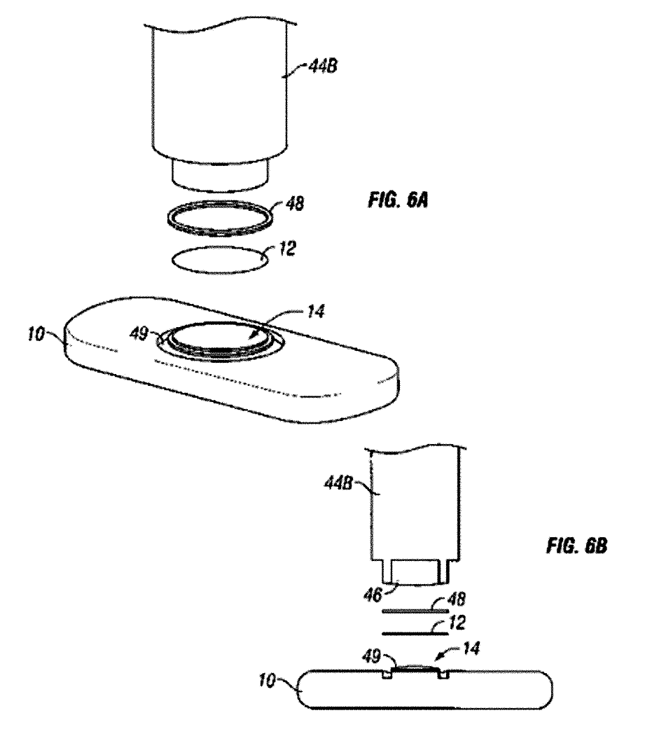

Figs. 6A and 6B are perspective and side cross-sectional views of a membrane adhesion process in an alternative embodiment, wherein the membrane 12 is sandwiched between the device body 10 and a plastic donut or disc 48. In this embodiment, the disc 48 is adapted to fit within a groove 49 formed in the device body 10, however not all embodiments require a groove for receiving the disc 48. In this embodiment, the sensing region 14 is raised from the surface of the device body 10 (see Fig. 6B) and includes a smooth, convexly curved surface, which is believed to minimize or eliminate wrinkling or bubbling of the membrane after adhesion. Preferably, the disc 48 is formed from a material substantially similar to the device body 10, or an insert formed therein (such as insert 40 of Figs. 4 and 5, not illustrated in Fig. 6), so as to optimize the adhesive joint to enable strong adhesion therebetween. The circular or non-circular disc 48 is sized with an outer periphery or diameter greater than or equal to the periphery or diameter of the membrane 12. A central portion of the disc 48 is cut out so as to allow exposure of at least a substantial portion of the sensing region 14 through the membrane 12. Thus, the central aperture of the disc 48 is sized smaller than the membrane, but large enough to expose the sensing region 14, for example an electrode system, through the membrane 12. In some embodiments, the thickness of the disc 48 is substantially the same as the depth of the groove 49 so as to provide a flush final assembly between the disc 48 and the device body. However, the disc need not lie flush with the device body in some embodiments. This configuration can be advantageous, for example, when the membrane 12 can benefit from added mechanical strength (from the disc 48) to support the membrane. It is noted that the disc 48 and associated hot die 44b can be provided in a variety of configurations as is also appreciated by one skilled in the art.

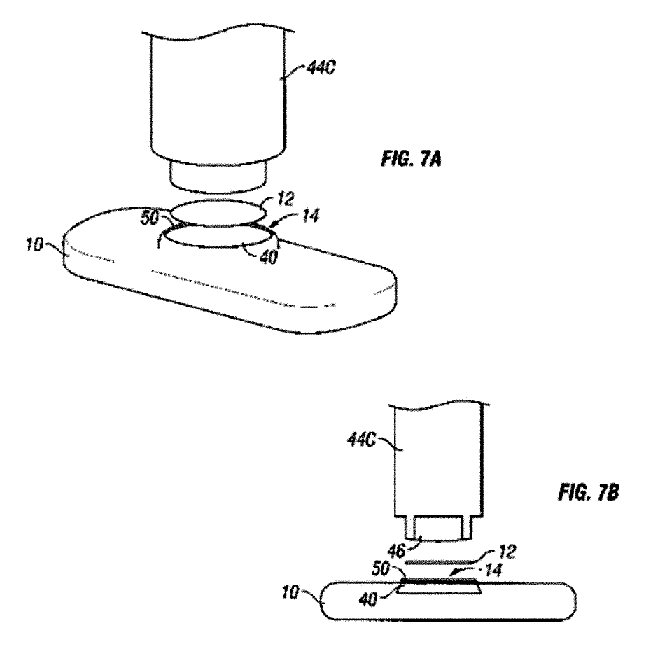

Figs. 7A and 7B are perspective and side cross-sectional views of a membrane adhesion process in another alternative embodiment, wherein the insert 40 includes a raised portion, also referred to as a ridge 50, substantially surrounding the periphery of the membrane 12. It is noted that in embodiments wherein the body is formed form a material substantially similar to the membrane system, the separate insert is not included and the membrane system 12 is adhered directly to the body 10, which can include a ridge 50. The hot die 44c, which includes an inset 46 (not to scale in the drawing), is configured to melt and/or mold the ridge 50 over the membrane 12 so as to securely seal and hold the membrane under the ridge 50. The die 44c preferably uses pressure and/or thermal energy to mold the ridge 50 over the membrane to mold the ridge 50 over the membrane system 12.

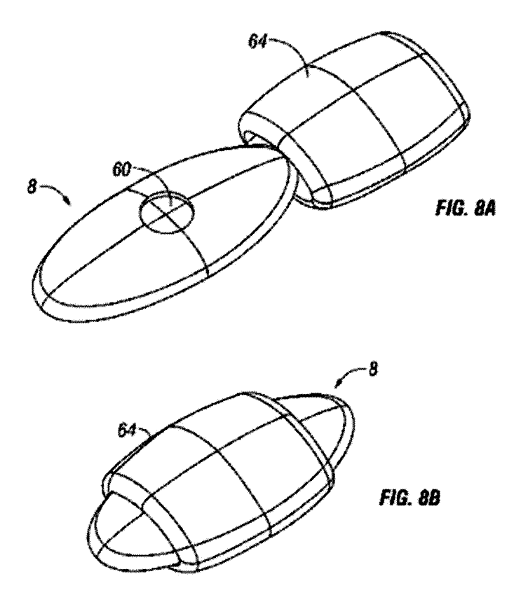

Figs. 8A to 11B are perspective views of some alternative configurations for membrane attachment with the preferred embodiments. The concepts described above can be partially or fully applied to these alternative configurations as described in more detail below. One skilled in the art appreciates these illustrations do not in any way limit other modifications to the systems and methods of the preferred embodiments.

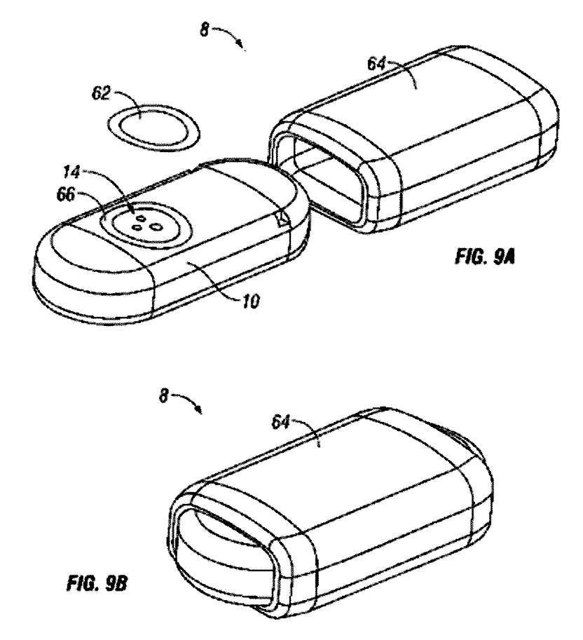

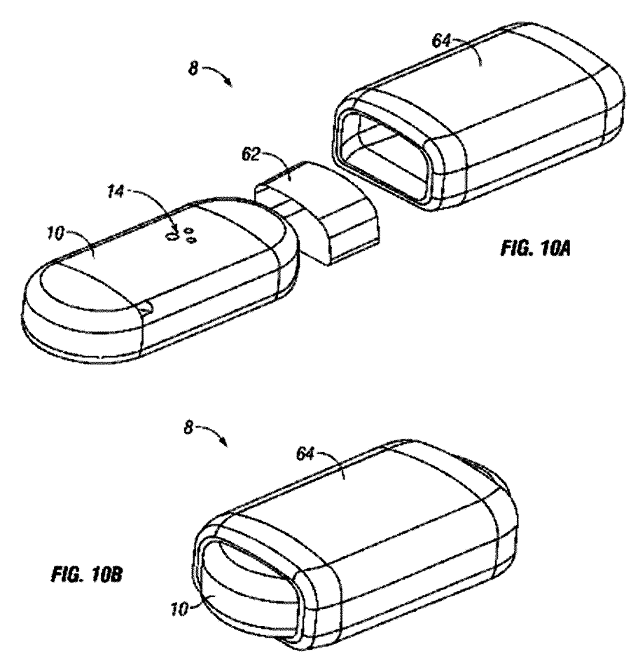

Figs. 8A and 8B are unassembled and assembled perspective views of one alternative embodiment of an analyte measuring device 8 including an inset portion 60 located thereon. In this embodiment, an inner membrane 62 (shown in Figs. 9 and 10), which can include, for example, a sensing membrane 30 and optionally additionally a cell impermeable domain 18, is applied directly into the inset portion 60 of the device 8. In some embodiments, the body can be formed from a substantially dissimilar material to the membrane system; in such embodiments, the inset can comprise an insert 40 from a substantially similar material to the membrane system. In some embodiments, a thermal bond can be used to adhere the inner membrane 62 to the inset 60. In some embodiments, a solvent bond or liquid adhesive can be used to adhere the inner membrane 62 to the inset 60. It is noted that a preferred embodiment is illustrated including the inner membrane 62 being at least substantially flush with the surface of the device 8 (or slightly higher) after the membrane adhesion process, such that the apex of the sensing region is substantially the apex of the sensor body (see co-pending U.S. Application No. 10/646,333 filed August 22, 2003 entitled, “OPTIMIZED SENSOR GEOMETRY FOR AN IMPLANTABLE GLUCOSE SENSOR.”) Subsequently, an outer membrane 64 slides or unrolls onto the smooth device surface. The outer membrane 64 can be secured by tension of the membrane’s elasticity around the device, by an adhesive, or the like, to ensure that slippage does not occur between the device and the outer membrane 64. The outer portion 64 can include, for example, a porous tissue anchoring material, biointerface membrane 28, and/or a cell disruptive domain 16 alone, such as described in more detail with reference to Figs. 2A to 2C.

Figs. 9A and 9B are unassembled and assembled perspective views of another alternative embodiment of an analyte measuring device 8 including a groove 66 located thereon. In this embodiment, an inner membrane 62 (for example, a sensing membrane 30 and optionally additionally a cell impermeable domain 18) is applied directly to the sensing region 14 and adhered at the groove 66. In some embodiments, the sensing region 14 is raised from the plane of the device body 10 and can include a curvature, as described in more detail elsewhere herein. It is noted that the inner membrane 62 can be adhered in any manner described herein with reference to the preferred embodiments. In some embodiment, an outer membrane 64 slides or unrolls onto the smooth device surface. However the outer membrane 64 can be adhered using any method described herein and/or other methods appreciated in the art. The outer membrane 64 can include, for example, a biointerface membrane 28 or a cell disruptive domain 16 alone, such as described in detail with reference to Figs. 2A to 2C.

Figs. 10A and 10B are unassembled and assembled perspective views of another alternative embodiment of an analyte measuring device 8, wherein an inner membrane 62 and outer membrane 64 are designed to be deposited on, slide over, or unroll onto a smooth device surface. In this embodiment, the inner membrane 62 is in the form of a sleeve that, after placement on the device surface, can be adhered using any of the techniques described with reference to the preferred embodiments. It is noted in this embodiments, that adhesion can optionally be required only at the exposed edges of the membrane. After attaching of the inner membrane 62, the outer membrane 64 slides over the device and can be held or adhered as described in more detail with reference to Figs. 8A to 9B.

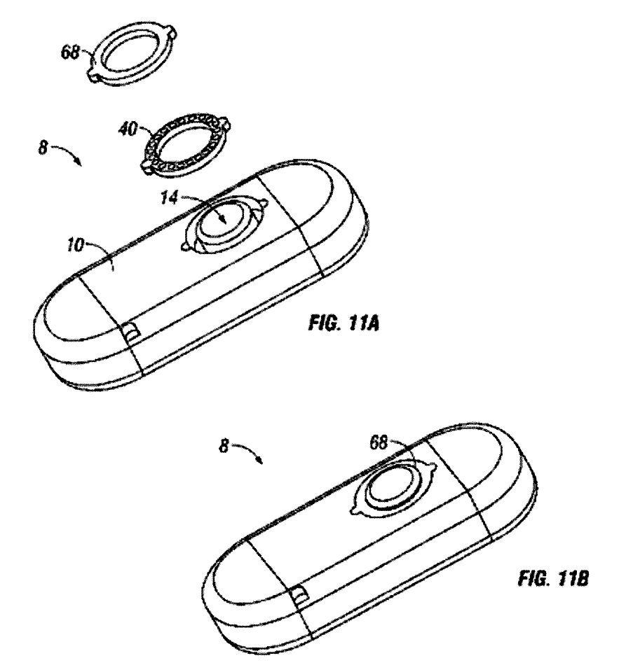

Figs. 11A and 11B are unassembled and assembled perspective views of another alternative embodiment of an analyte measuring device 8, wherein a membrane attachment mechanism includes a plastic insert 40 and a plastic disc 68 that press- or snap-fit into each other. In the illustrated embodiment, the insert 40 includes a plurality of male mating parts that are adapted to mate to female mating parts on the disc 68 (not shown). However, any chemical, mechanical, or combination chemical-mechanical attachment mechanism can be used herein. It is noted that a membrane system 12 (not shown here) is sandwiched between the insert 40 and disc 68 in a secure fashion by virtue of the mating parts and/or other attachment mechanism. Although not required, the mating insert 40 and ring 68 are advantageously designed such that the membrane system can be held securely therebetween prior to inserting the insert 40 and disc 68 subassembly into the device body 10 for final attachment. By enabling the membrane system to be securely held over the sensing region 14 prior to final attaching (for example, without inducing surface modification of the disc and insert), the membrane system can be tested for manufacturing purposes, or the like, prior to the subsequent attachment process 36. Final attachment includes securely attaching the insert 40 and disc 68 into the device body, using mechanical (for example, press- or snap-fit), thermal, chemical, or any combination of attachment techniques such as described in more detail elsewhere herein.

Alternatively, the insert 40 is built into the device and the disc 68 adapted to mate with the insert 40 within the device body 10. As yet another alternative, the insert 40 can be inserted into the device body, after which the membrane system and then the disc 68 securely attached or adhered thereto. It is appreciated by one skilled in the art that a variety of modifications are possible within the scope of the preferred embodiments.

Methods and devices that are suitable for use in conjunction with aspects of the preferred embodiments are disclosed in copending U.S. Patent Application 10/842,716 filed May 10, 2004 and entitled, “BIOINTERFACE MEMBRANES INCORPORATING BIOACTIVE AGENTS”; U.S. Patent Application 10/838,912 filed May 3, 2004 and entitled, “IMPLANTABLE ANALYTE SENSOR”; U.S. Application No. 10/789,359 filed February 26, 2004 and entitled, “INTEGRATED DELIVERY DEVICE FOR A CONTINUOUS GLUCOSE SENSOR”; U.S. Application No. 10/685,636 filed October 28, 2003 and entitled, “SILICONE COMPOSITION FOR BIOCOMPATIBLE MEMBRANE”; U.S. Application No. 10/648,849 filed August 22, 2003 and entitled, “SYSTEMS AND METHODS FOR REPLACING SIGNAL ARTIFACTS IN A GLUCOSE SENSOR DATA STREAM”; U.S. Application No. 10/646,333 filed August 22, 2003 entitled, “OPTIMIZED SENSOR GEOMETRY FOR AN IMPLANTABLE GLUCOSE SENSOR”; U.S. Application No. 10/647,065 filed August 22, 2003 entitled, “POROUS MEMBRANES FOR USE WITH IMPLANTABLE DEVICES”; U.S. Application No. 10/633,367 filed August 1, 2003 entitled, “SYSTEM AND METHODS FOR PROCESSING ANALYTE SENSOR DATA”; U.S. Application No. 09/916,386 filed July 27, 2001 and entitled “MEMBRANE FOR USE WITH IMPLANTABLE DEVICES”; U.S. Appl. No. 09/916,711 filed July 27, 2001 and entitled “SENSOR HEAD FOR USE WITH IMPLANTABLE DEVICE”; U.S. Appl. No. 09/447,227 filed November 22, 1999 and entitled “DEVICE AND METHOD FOR DETERMINING ANALYTE LEVELS”; U.S. Appl. No. 10/153,356 filed May 22, 2002 and entitled “TECHNIQUES TO IMPROVE POLYURETHANE MEMBRANES FOR IMPLANTABLE GLUCOSE SENSORS”; U.S. Appl. No. 09/489,588 filed January 21, 2000 and entitled “DEVICE AND METHOD FOR DETERMINING ANALYTE LEVELS”; U.S. Appl. No. 09/636,369 filed August 11, 2000 and entitled “SYSTEMS AND METHODS FOR REMOTE MONITORING AND MODULATION OF MEDICAL DEVICES”; and U.S. Appl. No. 09/916,858 filed July 27, 2001 and entitled “DEVICE AND METHOD FOR DETERMINING ANALYTE LEVELS,” as well as issued patents including U.S. 6,001,067 issued December 14, 1999 and entitled “DEVICE AND METHOD FOR DETERMINING ANALYTE LEVELS”; U.S. 4,994,167 issued February 19, 1991 and entitled “BIOLOGICAL FLUID MEASURING DEVICE”; and U.S. 4,757,022 filed July 12, 1988 and entitled “BIOLOGICAL FLUID MEASURING DEVICE.” The foregoing patent applications and patents are incorporated herein by reference in their entireties.

The preferred embodiments can be modified or combined with a variety of alternative membrane manufacture and attachment systems and methods. For example, in some embodiments, one or more domains of the membrane system can be deposited directly onto the sensing region using thin film techniques, such as spin coating, dip coating, wire-bar coating, blade coating, roller coating, solvent casting, screen printing, ink jet printing, pad printing, gravure printing, electrostatic spraying, and deposition methods, such as vacuum evaporation or electrical, chemical, screening, vapor deposition, or the like. In these embodiments, additional layers can be attached or otherwise adhered to the device using the systems and methods of the preferred embodiments. It is additionally noted that aspects of illustrated embodiments can be combined or modified in view of other embodiments described herein or appreciated by one skilled in the art, without departing from the spirit or scope of the preferred embodiments.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

All references cited herein are incorporated herein by reference in their entirety. To the extent publications and patents or patent applications incorporated by reference contradict the disclosure contained in the specification, the specification is intended to supersede and/or take precedence over any such contradictory material.

What is Claimed is:

1. An implantable analyte-measuring device, comprising:
a sensor body formed from a first material, wherein the sensor body comprises a sensing region for measuring an analyte; and
a membrane system configured to permit passage of the analyte at least partially therethrough, wherein the membrane system is adhered to the sensor body such that the membrane system substantially covers the sensing region.

2. The device of claim 1, wherein the first material comprises at least one material selected from the group consisting of plastics, metals, ceramics, and combinations thereof.

3. The device of claim 1, wherein the first material comprises a plastic material.

4. The device of claim 3, wherein the plastic material comprises a thermoset material.

5. The device of claim 4, wherein the thermoset material comprises an epoxy.

6. The device of claim 3, wherein the plastic material comprises a thermoplastic material.

7. The device of claim 1, wherein the sensor body further comprises an insert formed from a second material, wherein the insert is situated within the sensor body or on the sensor body at a location substantially within the sensing region or around the sensing region.

8. The device of claim 7, wherein the second material comprises a plastic material.

9. The device of claim 8, wherein the plastic material comprises a thermoplastic material.

10. The device of claim 8, wherein the plastic material comprises a thermoset material.

11. The device of claim 1, wherein the membrane system comprises a plastic film.

12. The device of claim 11, wherein the membrane system comprises a thermoplastic film or a thermoset film.

13. The device of claim 1, wherein the membrane is adhered to the body by application of heat.

14. The device of claim 1, wherein the membrane is adhered to the body by solvent welding.

15. The device of claim 1, wherein the membrane is adhered to the body by an adhesive.

16. The device of claim 1, wherein the membrane system is adhered to the body by application of pressure.

17. The device of claim 1, wherein the sensor body comprises a substantially curved surface.

18. The device of claim 17, wherein the sensing region extends outward from a portion of the sensor body.

19. The device of claim 18, wherein the sensing region comprises a convexly curved surface.

20. The device of claim 1, wherein the membrane system comprises at least one component selected from the group consisting of a cell disruptive domain, a cell impermeable domain, a resistance domain, an enzyme domain, an interference domain, and an electrolyte domain.

21. The device of claim 1, wherein the sensing region comprises a sensing mechanism selected from the group consisting of enzymatic, chemical, physical, optical, electrochemical, spectrophotometric, polarimetric, amperometric, calorimetric, and radiometric.

22. The device of claim 1, further comprising a disc adapted to adhere at least a periphery of the membrane system to the sensor body.

23. The device of claim 1, wherein the sensor body further comprises a ridge substantially surrounding a periphery of the membrane system when the membrane system is placed over the sensing region.

24. The device of claim 1, further comprising an inset portion within the sensor body, wherein the inset portion is configured to receive the membrane system.

25. The device of claim 1, further comprising a groove surrounding the sensing region.

26. The device of claim 1, wherein the membrane system is adhered at its periphery to the sensor body with sufficient strength to withstand in vivo cellular forces.

27. A method for manufacturing an analyte-measuring device comprising a sensing region for measuring the analyte, the method comprising:
providing a membrane system;
placing the membrane system on the analyte measuring device so as to cover the sensing region; and
adhering at least a peripheral portion of the membrane system to the analyte measuring device such that analyte transport occurs only by diffusion through the membrane system.

28. The method of claim 27, wherein the adhering step comprises adhering the membrane system to the device at a periphery of the membrane system, wherein a resulting bond between the device and the membrane system is sufficient strength to withstand in vivo cellular forces.

29. The method of claim 27, wherein the adhering step comprises adhering using thermal energy.

30. The method of claim 29, wherein the thermal energy comprises ultrasonic welding.

31. The method of claim 27, wherein the adhering step comprises adhering using solvent welding.

32. The method of claim 27, wherein the adhering step comprises applying an adhesive.

33. The method of claim 27, wherein the adhering step comprises applying pressure.

34. The method of claim 27, wherein the adhering step comprises applying a hot die over the membrane system.

35. The method of claim 27, wherein the adhering step comprises attaching a disc to the device so as to secure the membrane system therebetween, wherein the disc is adapted to be placed over the membrane system and is configured to cover at least a periphery of the membrane system.

36. The method of claim 27, wherein the device comprises a portion with a ridge configured to surround the membrane system, and wherein the adhering step molds the ridge over the membrane system.

37. An implantable glucose-measuring device, comprising:
a sensor body comprising a thermoset material, wherein the sensor body comprises a sensing region for measuring glucose;
an insert comprising a thermoplastic material, wherein the insert is situated within the sensor body at a location substantially within the sensing region or surrounding the sensing region; and
a membrane system permitting passage of the analyte at least partially therethrough, wherein the membrane system is adhered to the sensor body on the insert such that the membrane system substantially covers the sensing region.

38. The device of claim 37, wherein the membrane system is adhered to the insert by application of heat.

39. The device of claim 37, wherein the membrane system is adhered to the insert such that the periphery of the membrane system is sealed to the insert.
